(19) United States
(12) Patent Application Publication
(10) Pub. No.: US 2016/0000359 A1
(43) Pub. Date: Jan. 7, 2016

(54) Human Body Movement State Monitoring Method And Device

(71) Applicant: GOERTEK INC, Shandong (CN)

(72) Inventors: Bo Li, Welfang City, ShanDong Province (CN); Fupo Wang, Welfang City, ShanDong Province (CN); Na Li, Welfang City, ShanDong Province (CN)

(21) Appl. No.: 14/770,731

(22) PCT Filed: Dec. 25, 2014
(86) PCT No.: PCT/CN2014/001178

§ 371 Date: Aug. 26, 2015

(30) Foreign Application Priority Data
Dec. 31, 2013 (CN) 201310754081.X

(51) Int. Cl. A61B 5/11; A61B 5/00

(52) U.S. Cl. CPC A61B 5/1123; A61B 5/4806; A61B 5/7225; A61B 5/742

(57) ABSTRACT

The present invention provides a human body movement state monitoring method and device. The method comprises the following steps performed repeatedly: obtaining acceleration signals having a set sampling time period from output of a triaxial acceleration sensor worn on a human body, and calculating the energy and average power of the acceleration signals; determining a human body movement state according to the average power of the acceleration signals, and if the average power of the acceleration signals is more than a predetermined fierce movement threshold, determining that the human body is in a fierce movement state, if the average power of the acceleration signals is less than a predetermined sleeping threshold, determining that the human body is in a sleeping state, if the average power of the acceleration signals is less than the fierce movement threshold and is more than the sleeping threshold, determining that the human body is in a light movement state; if the human body is in the fierce movement state, further determining whether the acceleration signals have quasi-periodicity, if the acceleration signals do not have quasi-periodicity, determining that the human body is in an irregular fierce movement state, if the acceleration signals have quasi-periodicity, determining that the human body is in a regular fierce movement state. The method can automatically, comprehensively, round-the-clock, accurately monitor various movement states of a person.

### FIELD OF THE INVENTION

The present invention relates to the field of sports equipment, in particular, relates to a human body movement state monitoring method and device.

### BACKGROUND OF THE INVENTION

With the continuous development of social economy, material living standard is gradually improved, and at the same time, health of ones own is more and more concerned, and a variety of sports schemes for fitness are customized for ones own. Therefore, various devices for monitoring sports schemes occur.

Pedometer is a device capable of calculating the walk or run step number of its wearer. However, pedometer merely has a rather single function, which cannot monitor other movement forms and sleeping of a person. There are also some devices capable of monitoring various forms of movement of a person comprehensively, which require the intervention of human operation, and fail to monitor various movement conditions and sleeping conditions of the person automatically, comprehensively, and round-the-clock. Meanwhile, sleeping conditions are very helpful for determining the health conditions of the person, During sleeping, the person may make some abnormal movements such as turning, scratching, scaring, etc. If such abnormal movements occur rather frequently, it indicates that the person's sleep quality is not good enough. Therefore, both the monitoring of person's sleeping conditions and normal movement conditions are helpful for improving one's fitness schemes.

### SUMMARY OF THE INVENTION

The present invention is aimed to solve the problems existing in the aforesaid prior art, with an object for providing a human body movement state monitoring method and device, the method and device can automatically, comprehensively, round-the-clock, accurately monitor various movement states of a person, thus provide basis for improving one's fitness schemes.

For achieving aforesaid object, one aspect of the present invention provides a human body movement state monitoring method, the method comprising the following steps performed repeatedly:
a) obtaining acceleration signals having a set sampling time period from output of a triaxial acceleration sensor worn on a human body, and calculating the energy and average power of the acceleration signals;
b) determining a human body movement state according to the average power of the acceleration signals, and if the average power of the acceleration signals is more than a predetermined fierce movement threshold, determining that the human body is in a fierce movement state, if the average power of the acceleration signals is less than a predetermined sleeping threshold, determining that the human body is in a sleeping state, if the average power of the acceleration signals is less than the fierce movement threshold and is more than the sleeping threshold, determining that the human body is in a light movement state;
c1) if the human body is in the sleeping state, accumulating the time periods of the acceleration signals into the total time period of the sleeping state, accumulating the energy of the acceleration signals into the total energy of the sleeping state, counting up the time periods of acceleration signals which have intensity more than a predetermined intensity threshold, and accumulating the counted time periods into the total time period of sleeping abnormal movements, and setting the sampling time period of acceleration signals as the sampling time period of the sleeping state, then returning to step a);
c2) if the human body is in the light movement state, accumulating the time periods of the acceleration signals into the total time period of the light movement state, accumulating the energy of the acceleration signals into the total energy of the light movement state, and setting the sampling time period of acceleration signals as the sampling time period of the light movement state, then returning to step a);
c3) if the human body is in the fierce movement state, further determining whether the acceleration signals have quasi-periodicity, if the acceleration signals do not have quasi-periodicity, determining that the human body is in an irregular fierce movement state, if the acceleration signals have quasi-periodicity, determining that the human body is in a regular fierce movement state;
d1) if the human body is in the irregular fierce movement state, accumulating the time periods of the acceleration signals into the total time period of the irregular fierce movement state, accumulating the energy of the acceleration signals into the total energy of the irregular fierce movement state, and setting the sampling time period of acceleration signals as the sampling time period of the fierce movement state, then returning to step a);
d2) if the human body is in the regular fierce movement state, accumulating the time periods of the acceleration signals into the total time period of the regular fierce movement state, accumulating the energy of the acceleration signals into the total energy of the regular fierce movement state, calculating movement step number according to the acceleration signals, and accumulating the movement step number into the total movement step number, and setting the sampling time period of acceleration signals as the sampling time period of the fierce movement state, then returning to step a). Preferably, the average power P of the acceleration signals can be calculated from the following formula:

$$P = \frac{1}{N} {\sum }_{i = 1}^{N} {( {a}_{i} - {a}_{0} )}^{2}$$

wherein aᵢ is the No. i value of the acceleration signals, N is the length of the acceleration signals, and 1≦i≦N, a₀ is the average value of the acceleration signals,

$${a}_{0} = \frac{1}{N} {\sum }_{i = 1}^{N} {a}_{i} .$$

Preferably, the determining step of the quasi-periodicity of the acceleration signals can comprise:
performing high-pass filtering on the acceleration signals;
performing pitch detection on the high-pass filtered acceleration signals;
setting a low-pass or band-pass filter by using the pitch obtained by the pitch detection as cut-off frequency, and using the low-pass or band-pass filter to perform low-pass or band-pass filtering on corresponding high-pass filtered acceleration signals;
obtaining extreme value points of the acceleration signals in the low-pass or band-pass filtered acceleration signals and removing interfering extreme value points in the extreme value points of the acceleration signals, so as to obtain effective extreme value points in the low-pass or band-pass filtered acceleration signals;
calculating time gaps between adjacent effective extreme value points, obtaining a time gap sequence, and calculating differences between adjacent time gaps in the time gap sequence, obtaining a time gap difference sequence, and if each of a continuous predetermined number of time gap differences in the time gap difference sequence is less than a predetermined period threshold, determining that the acceleration signals have quasi-periodicity, otherwise, determining that the acceleration signals do not have quasi-periodicity.

Further preferably, the step of calculating movement step number according to the acceleration signals can comprise:

counting the effective extreme value points in the low-pass or band-pass filtered acceleration signals having quasi-periodicity, the number of the effective extreme value points being movement step number.

Further preferably, a displacement signal can be obtained by double integral of the acceleration signals on time.

The method of performing pitch detection on the high-pass filtered acceleration signals can comprise one or more of autocorrelation function method, cepstrum method, linear predictive coding method and average magnitude difference function method.

Preferably, performing pitch detection on the high-pass filtered acceleration signals can comprise: attenuating the signals with a filter that attenuates signals with an incrementing degree from low frequency to high frequency; obtaining the autocorrelation function ρ(τ) of the attenuated signals from the following formula:

$$ρ ( τ ) = \frac{{\sum }_{n = 1}^{N} a ( n ) a ( n - τ )}{\sqrt{{\sum }_{n = 1}^{N} {a}^{2} ( n ) {\sum }_{n = 0}^{N - 1} {a}^{2} ( n - τ )}}$$

wherein a(n) is the No. n value of the attenuated signals, N is the length of the signals, and 1≦n≦N, τ is a delay time, ρ(τ) is normalized autocorrelation function of the signals; calculating the value of τ corresponding to the maximal value of ρ(τ), and the reciprocal of the τ value is the pitch of the signals.

Wherein, removing the interfering extreme value points from the extreme value points of the acceleration signals comprises: filtering out the interfering extreme value points from the extreme value points of the acceleration signals through a time gap; alternatively, filtering out the interfering extreme value points from the extreme value points of the acceleration signals through a time gap and a magnitude value.

Preferably, the interfering extreme value points may comprise such an extreme value point of the acceleration signals that the time gap between the extreme value point of the acceleration signals and the previous extreme value point of the acceleration signals is less than a predetermined threshold; or the interfering extreme value points may comprise extreme value points of the acceleration signals, whose magnitude values are not maximal, among each group of extreme value points of the acceleration signals with time gaps continuously less than a predetermined threshold.

Preferably, the human body movement state monitoring method also can comprise optionally displaying the total time period of the sleeping state, the total energy of the sleeping state, the total time period of the sleeping abnormal movements, the total time period of the light movement state, the total energy of the light movement state, the total time period of the irregular fierce movement state, the total energy of the irregular fierce movement state, the total time period of the regular fierce movement state, the total energy of the regular fierce movement state and the total step number of movement.

According to another aspect of the present invention, a human body movement state monitoring device is provided, which comprises: a triaxial acceleration sensor, an acceleration signal obtaining unit, a calculating unit, a human body movement state determining unit, a sleeping abnormal movement statistical unit, a sampling time period setting unit, a storage unit, a quasi-periodicity determining unit, and a step counting unit.

The acceleration signal obtaining unit obtains acceleration signals having a set sampling time period from output of the triaxial acceleration sensor worn on a human body, and the calculating unit calculates the energy and average power of the acceleration signals;

the human body movement state determining unit determines a human body movement state according to the average power of the acceleration signals, and if the average power of the acceleration signals is more than a predetermined fierce movement threshold, determines that the human body is in a fierce movement state, if the average power of the acceleration signals is less than a predetermined sleeping threshold, determines that the human body is in a sleeping state, if the average power of the acceleration signals is less than the fierce movement threshold and is more than the sleeping threshold, determines that the human body is in a light movement state;

if the human body movement state determining unit determines that the human body is in the sleeping state, accumulates the time periods of the acceleration signals into the total time period of the sleeping state, accumulates the energy of the acceleration signals into the total energy of the sleeping state, the sleeping abnormal movement statistical unit counts up the time periods of acceleration signals which have intensity more than a predetermined intensity threshold, and accumulates the counted time periods into the total time period of sleeping abnormal movements, the sampling time period setting unit sets the sampling time period of acceleration signal as the sampling time period of the sleeping state, the storage unit stores the total time period of the sleeping state, the total energy of the sleeping state and the total time period of the sleeping abnormal movements;

if the human body movement state determining unit determines that the human body is in the light movement state, accumulates the time periods of the acceleration signals into the total time period of the light movement state, accumulates the energy of the acceleration signals into the total energy of the light movement state, and the sampling time period setting unit sets the sampling time period of acceleration signal as the sampling time period of the light movement state, the storage unit stores the total time period of the light movement state and the total energy of light movement state;

if the human body movement state determining unit determines that the human body is in the fierce movement state, then the quasi-period determining unit determines whether the acceleration signals have quasi-periodicity, and if determining that the acceleration signals do not have quasi-periodicity, then the human body movement state determining unit determines that the human body is in an irregular fierce movement state, and if the quasi-period determining unit determines that the acceleration signals have quasi-periodicity, then the human body movement state determining unit determines that the human body is in a regular fierce movement state;

if the human body movement state determining unit determines that the human body is in the irregular fierce movement state, then accumulates the time periods of the acceleration signals into the total time period of the irregular fierce movement state, accumulates the energy of the acceleration signals into the total energy of the irregular fierce movement state, and the sampling time period setting unit sets the sampling time period of acceleration signals as the sampling time period of the fierce movement state, the storage unit stores the total time period of the irregular fierce movement state and the total energy of the irregular fierce movement state;

if the human body movement state determining unit determines that the human body is in the regular fierce movement state, then accumulates the time periods of the acceleration signals into the total time period of the regular fierce movement state, accumulates the energy of the acceleration signals into the total energy of the regular fierce movement state, and the step counting unit calculates movement step number according to the acceleration signals, and accumulates the movement step number into the total movement step number, the sampling time period setting unit sets the sampling time period of acceleration signal as the sampling time period of fierce movement state, the storage unit stores the total time period of the regular fierce movement state, the total energy of the regular fierce movement state and the movement step number.

Preferably, the human body movement state monitoring device may further comprise a display unit for optionally displaying the total time period of the sleeping state, the total energy of the sleeping state, the total time period of the sleeping abnormal movements, the total time period of the light movement state, the total energy of the light movement state, the total time period of the irregular fierce movement state, the total energy of the irregular fierce movement state, the total time period of the regular fierce movement state, the total energy of the regular fierce movement state and the total step number of movement.

It is known from the above description and practice that the human body movement state monitoring method and device of the present invention can automatically, comprehensively, round-the-clock, accurately monitor various movement states (including sleeping state) of a person, can measure the quality of human sleep quantitatively, measure their movement step number during walking and running accurately, and can measure their movement level and energy consumption conditions quantitatively.

The aforesaid description is merely the summary of technical solution of the present invention, for understanding the technical means of the present invention more clearly, it can be carried out according to the disclosure of specification, and for making aforesaid and other objectives, features and advantages of the present invention more apparent to be understood, embodiments of the present invention are listed as follows.

### BRIEF DESCRIPTION OF DRAWINGS

By reading the detailed description of the preferable embodiment hereinafter, all the other features and advantages will be apparent to the general skilled in the art. The attached drawings are merely for the purpose of demonstrating preferable embodiment, rather than deemed as a limitation of the present invention. Among the attached drawings:

FIG. 1 is a signal graph, demonstrating the example of acceleration signals produced by a triaxial acceleration sensor in three directions during sleeping of its wearer;

FIG. 2 is a signal graph, demonstrating the example of acceleration signals produced by the triaxial acceleration sensor in three directions during light movement of its wearer;

FIG. 3 is a signal graph, demonstrating the example of acceleration signals produced by the triaxial acceleration sensor in three directions during irregular fierce movement of its wearer;

FIG. 4 is a signal graph, demonstrating the example of acceleration signals produced by the triaxial acceleration sensor in three directions during regular fierce movement of its wearer;

FIG. 5 is a block diagram, demonstrating a human body movement state monitoring method according to one embodiment;

FIG. 6a is a signal graph, demonstrating representative normalized acceleration signals having a predetermined length output from the triaxial acceleration sensor;

FIG. 6b is a signal graph, demonstrating high-pass filtered acceleration signals;

FIG. 6c is a signal graph, demonstrating low-pass filtered acceleration signals;

FIG. 6d is a signal graph, demonstrating an example of extreme value points of the low-pass filtered acceleration signals;

FIG. 7 demonstrating an example of frequency response curve of a filter that attenuates signals with an incrementing degree from low frequency to high frequency;

FIG. 8 is a signal graph, demonstrating another example of extreme value points of low-pass filtered monoaxial acceleration signals;

FIG. 9 is a block diagram, demonstrating a human body movement state monitoring device according to one embodiment;

FIG. 10 illustratively demonstrates a block diagram of a server for carrying out the method according to the present invention; and

FIG. 11 illustratively demonstrates a storage unit for maintaining or carrying the program codes for achieving the method according to the present invention.

### DETAILED DESCRIPTION OF THE INVENTION

The present invention will be described in details in combination with the attached drawings and specific examples.

In the following description, certain illustrative examples of the present invention are only described by way of illustration. Undoubtedly, one of ordinary skilled in the art may recognize that the examples can be amended by using a variety of different ways without departing from the spirit and scope of the present invention. Accordingly, the attached drawings and descriptions are illustrative in nature, and not intended to limit the protection scope of the claims. In the present specification, the same reference numerals denote the same or similar parts.

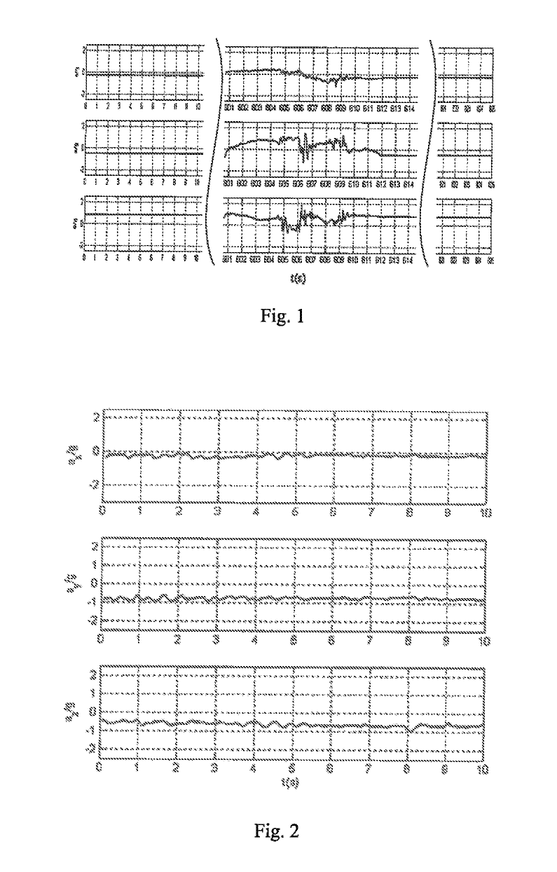

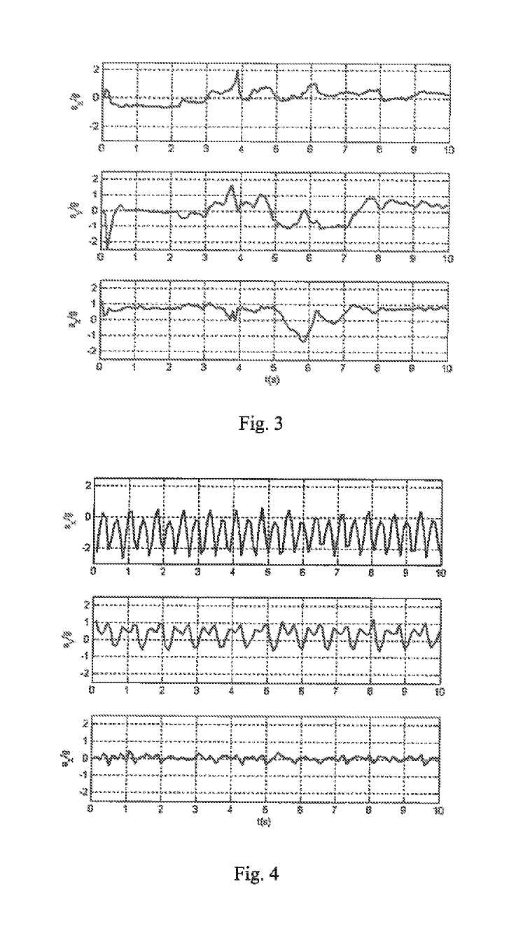

The human body movement state monitoring method of the present invention is carried out by using a device having a triaxial acceleration sensor. FIGS. 1-4 are signal graphs, respectively demonstrating the example of acceleration signals produced by the triaxial acceleration sensor in three directions during sleeping (including light sleep, deep sleep), light movements (such as typewriting, unconscious human body shaking, etc.), irregular fierce movements (manual labor, playing basketball, etc.) and regular fierce movements (walking, running, jumping rope, gym body building, etc.) of its wearer. As shown in FIGS. 1-4, generally speaking, the orientation of the device comprising the triaxial acceleration sensor in use is not unchanged, thus, during sleeping, light movements and irregular fierce movements, the triaxial output signals of the triaxial acceleration sensor are almost similar, while during regular fierce movements, signal intensity in a certain direction is stronger. In any case, the acceleration signal with the highest energy among the triaxial output of the triaxial acceleration sensor can be selected to measure movement conditions accurately and representatively. Therefore, in the description of the present invention, the acceleration signals output from the triaxial acceleration sensor can denote the acceleration signal with the highest energy among the triaxial output, but the present invention is not limited to this, and also may denote acceleration signals after triaxial output is fused in either way. Alternatively, it can also be, after obtaining corresponding measurement amount from each monoaxial acceleration signal of triaxial acceleration sensor, performing weighting and averaging in a certain weighting way, and finally obtaining a total measurement amount.

As shown in FIGS. 1-3, during sleeping, light movements and irregular fierce movements, the common feature of the acceleration signals output from the triaxial acceleration sensor is not having quasi-periodicity, therefore, it is possible to quantitatively measure the movement amount of these movement manners by measuring the duration and total energy of these signals. During the regular fierce movements, the feature of the acceleration signals output from the triaxial acceleration sensor is having quasi-periodicity, therefore, in addition to capable of measuring its duration and total energy, it is also able to further measure its period number, the period number corresponding to the step number of running, the number of jumping, the number of push-pull etc., and in the present invention, these amounts are referred to as movement step number. In another aspect, during sleeping, light movements and irregular (regular) fierce movements, different feature of the acceleration signals output from the triaxial acceleration sensor is different fierce degree of the signal change, which not only presented in the intensity scale of the acceleration signals, but also presented in the time scale. For example, during sleeping, the acceleration signals during most time are very small and gentle, the time with abnormal movements occurring during sleeping (For example, in FIG. 1 the turning-over movements occurring during the time period of 600˜610 seconds) accounts for a very small ratio of whole sleeping time.

Therefore, when analyzing the acceleration signals produced during sleeping, sampling length of signal should be relatively long, which not only facilitates for reducing computation amount, improving analyzing speed, but also makes abnormal movements during sleeping separable from the movements during light movements and fierce movements, this is because, since the time of abnormal movements accounts for a relatively small ratio of whole sleeping time, the contribution of abnormal movements of sleeping to the average power is negligible. Similarly, when analyzing the acceleration signals produced during light movements and fierce movements, sampling length should be different, for reducing computation amount, improving analyzing speed and also not enabling corresponding acceleration signals to lose feature.
One embodiment of the present invention provides a human body movement state monitoring method. The method comprises the following steps performed repeatedly:
step a) obtaining acceleration signals having a set sampling time period from output of a triaxial acceleration sensor worn on a human body, and calculating the energy and average power of the acceleration signals, turning to step b);
step b) determining a human body movement state according to the average power of the acceleration signals, and if the average power of the acceleration signals is more than a predetermined fierce movement threshold, it is determined that the human body is in a fierce movement state, turning to step c3), if the average power of the acceleration signals is less than a predetermined sleeping threshold, it is determined that the human body is in a sleeping state, turning to step c1); if the average power of the acceleration signals is less than the fierce movement threshold and is more than the sleeping threshold, it is determined that the human body is in a light movement state, turning to step c2);
step c1) if the human body is in the sleeping state, accumulating the time periods of the acceleration signals into the total time period of the sleeping state, accumulating the energy of the acceleration signals into the total energy of the sleeping state, counting up the time periods of acceleration signals which have intensity more than a predetermined intensity threshold, and accumulating the counted time periods into the total time period of sleeping abnormal movements, and setting the sampling time period of acceleration signals as the sampling time period of the sleeping state, then returning to step a);
step c2) if the human body is in the light movement state, accumulating the time periods of the acceleration signals into the total time period of the light movement state, accumulating the energy of the acceleration signals into the total energy of the light movement state, and setting the sampling time period of acceleration signals as the sampling time period of the light movement state, then returning to step a);
step c3) if the human body is in the fierce movement state, further determining whether the acceleration signals have quasi-periodicity, if the acceleration signals do not have quasi-periodicity, it is determined that the human body is in an irregular fierce movement state, turning to step d1); if the acceleration signals have quasi-periodicity, it is determined that the human body is in a regular fierce movement state, turning to step d2);
step d1) if the human body is in the irregular fierce movement state, accumulating the time periods of the acceleration signals into the total time period of the irregular fierce movement state, accumulating the energy of the acceleration signals into the total energy of the irregular fierce movement state, and setting the sampling time period of acceleration signal as the sampling time period of the fierce movement state, then returning to step a);
step d2) if the human body is in the regular fierce movement state, accumulating the time periods of the acceleration signals into the total time period of the regular fierce movement state, accumulating the energy of the acceleration signals into the total energy of the regular fierce movement state, calculating movement step number according to the acceleration signals, and accumulating the movement step number into the total movement step number, and setting the sampling time period of acceleration signals as the sampling time period of the fierce movement state, then returning to step a).

The reference sign added to each embodiment of the present invention is merely to facilitate demonstrating the operation order of a preferable solution, but does not limit specific operation sequence of each step strictly.

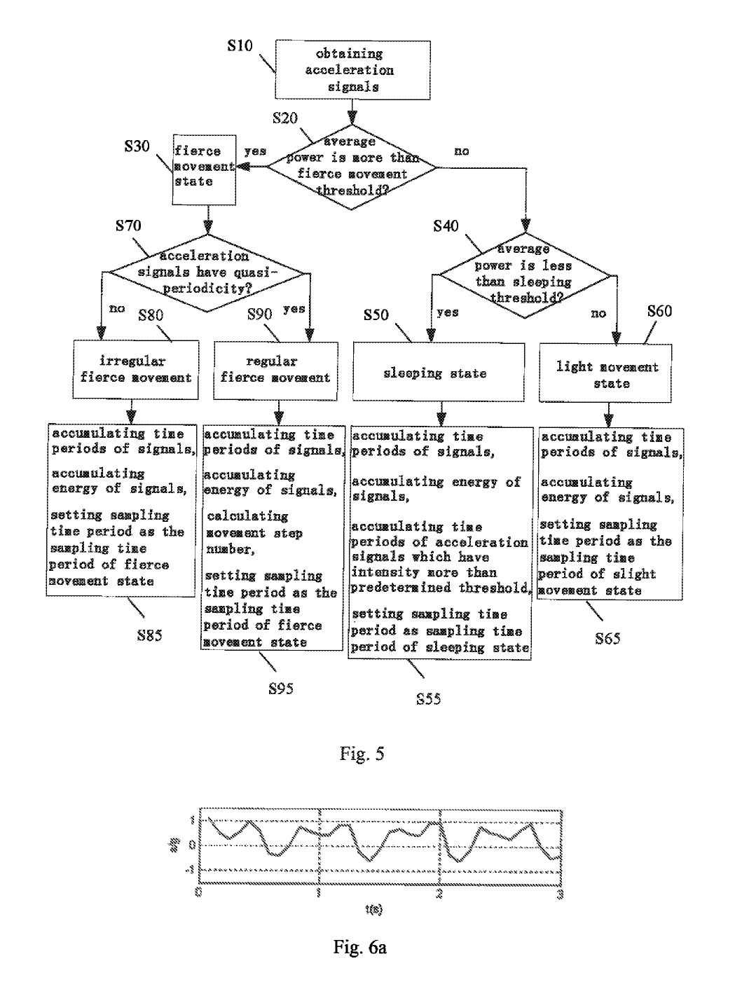

FIG. 5 is a block diagram, demonstrating a human body movement state monitoring method according to one embodiment. As shown in FIG. 5, the human body movement state monitoring method according to the embodiment comprises the following steps performed repeatedly:

firstly, in step S10, obtaining acceleration signals having a set sampling time period from output of a triaxial acceleration sensor worn on a human body, and calculating the energy and average power of the acceleration signals.

Preferably, the average power P of the acceleration signals can be calculated from the following formula:

$$P = \frac{1}{N} {\sum }_{i = 1}^{N} {( {a}_{i} - {a}_{0} )}^{2}$$

wherein aᵢ is the No. i value of the acceleration signals, N is the length of the acceleration signals, and 1≦i≦N, a₀ is the average value of the acceleration signals,

$${a}_{0} = \frac{1}{N} {\sum }_{i = 1}^{N} {a}_{i} .$$

subsequently, determining a human body movement state according to the average power of the acceleration signals. For example, in step S20, determining whether the average power of the acceleration signals is more than a predetermined fierce movement threshold, and if the average power of the acceleration signals is more than the predetermined fierce movement threshold, it is determined that the human body is in a fierce movement state (step S30), otherwise, in step S40, determining whether the average power of the acceleration signals is less than a predetermined sleeping threshold, and if the average power of the acceleration signals is less than the predetermined sleeping threshold, it is determined that the human body is in a sleeping state (step S50), if the average power of the acceleration signals is less than the fierce movement threshold and is more than the sleeping threshold, it is determined that the human body is in a light movement state (step S60). Wherein, the fierce movement threshold and the sleeping threshold can be obtained according to experiments, and can be adjusted.

If the human body is in the sleeping state, then in step S55, accumulating the time periods of the acceleration signals into the total time period of the sleeping state, accumulating the energy of the acceleration signals into the total energy of the sleeping state, counting up the time periods of acceleration signals which have intensity more than a predetermined intensity threshold, and accumulating the counted time periods into the total time period of sleeping abnormal movements, and setting the sampling time period of acceleration signals as the sampling time period of the sleeping state, then returning to step S10. It should be noted that, the time period of acceleration signals which has intensity more than a predetermined intensity threshold denotes such a period of time that within this period of time, the intensity size of the acceleration signals is more than the predetermined intensity threshold. This period of time is the time of abnormal movements of sleeping, during which abnormal movements of sleeping such as turning-over, scaring, spasm etc. occur, and through analyzing the ratio of total time period of abnormal movement time of sleeping accounting for total time period of sleeping, sleeping quality can be analyzed quantitatively, and when the ratio is very small, it denotes that the sleeping is deep sleeping, when the ratio is relatively large, it denotes that the sleeping is light sleeping. In addition, the sampling time period of sleeping state can be determined according to experiments, such as 5˜10 minutes.

If the human body is in the light movement state, then in step S65, accumulating the time periods of the acceleration signals into the total time period of the light movement state, accumulating the energy of the acceleration signals into the total energy of the light movement state, and setting the sampling time period of acceleration signals as the sampling time period of the light movement state, then returning to step S10. The sampling time period of light movement state can be determined according to experiment, such as 1 minute.

If the human body is in the fierce movement state, then in step S70 further determining whether the acceleration signals have quasi-periodicity, if the acceleration signals do not have quasi-periodicity, it is determined that the human body is in an irregular fierce movement state (step S80), if the acceleration signals have quasi-periodicity, it is determined that the human body is in a regular fierce movement state (step 90).

Wherein, the determination of the quasi-periodicity of the acceleration signals can comprise the following steps □ to □:

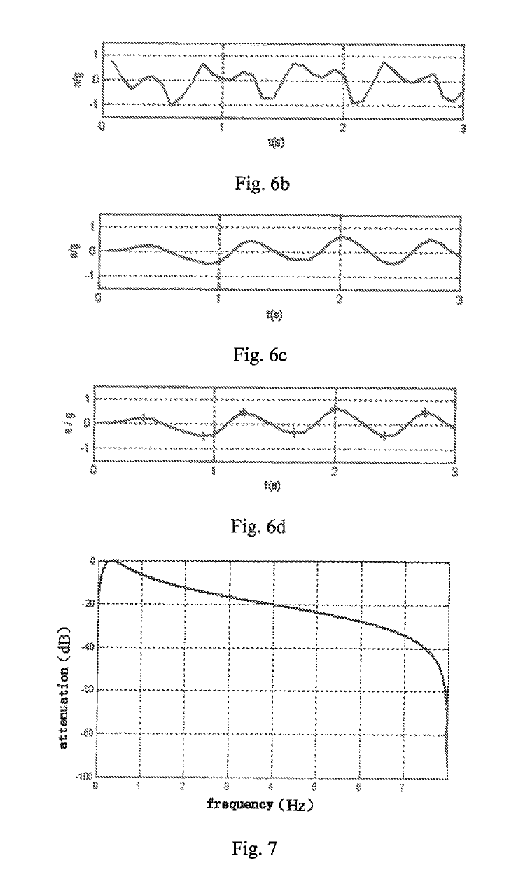

Step □ performing high-pass filtering on the acceleration signals. Since the acceleration signals output from the triaxial acceleration sensor generally comprise DC component, and the existence of the DC component would interfere with the analyzing of acceleration signals, the DC component is removed from the acceleration signals through high-pass filtering. FIG. 6a is a signal graph, demonstrating the representative normalized acceleration signal a/g having a predetermined length output from the triaxial acceleration sensor, wherein, a denotes acceleration, g denotes gravity acceleration. FIG. 6b is a signal graph, demonstrating high-pass filtered acceleration signals. It can be seen from FIG. 6b that after high-pass filtering, the acceleration signals only comprise AC component.

Step □ performing pitch detection on the high-pass filtered acceleration signals. The acceleration signals may comprise various frequency components corresponding to different body rhythmic movements, such as pitch component, frequency multiplication component and other high frequency components. FIG. 7 is schematic diagram of spectrum of acceleration signals. Wherein, pitch component is related to most fundamental rhythmic movements, and determining quasi-periodicity of signals according to the pitch component would be more accurate. For obtaining the acceleration signals only consisting of the pitch component, high frequency component in the acceleration signals is required to be filtered out. And in order to filter high frequency component, frequency of pitch component is required to be measured roughly, so as to configure a suitable filter for filtering high frequency component outside of the pitch component.

There are various methods for pitch detection, e.g., conventional methods in voice signal pitch detection such as autocorrelation function method, cepstrum method, linear predictive coding method, average magnitude difference function method can be used. Preferably, autocorrelation function method can be used.

Specifically, the high-pass filtered acceleration signals are attenuated with a filter that attenuates signal energy with an incrementing degree from low frequency to high frequency, to suppress high frequency component in the acceleration signals, so as to highlight the pitch component in the monoaxial acceleration signals, and reduce the error of obtained pitch. FIG. 7 shows an example of frequency response curve of a filter that attenuates signal energy with an incrementing degree from low frequency to high frequency. After acceleration signals are attenuated with the filter, low-frequency component in the signals is attenuated relatively slightly, while high frequency component is attenuated relatively largely. Thus, when further using autocorrelation function method to obtain pitch of the monoaxial acceleration signals filtered as such, the obtained pitch is relatively accurate.

Then, the autocorrelation function ρ(τ) of the attenuated signals is obtained from the following formula:

$$ρ ( τ ) = \frac{{\sum }_{n = 1}^{N} a ( n ) a ( n - τ )}{\sqrt{{\sum }_{n = 1}^{N} {a}^{2} ( n ) {\sum }_{n = 0}^{N - 1} {a}^{2} ( n - τ )}}$$

wherein a(n) is the No. n value of the attenuated signals, N is the length of the signals, and 1≦n≦N, τ is a delay time, ρ(τ) is normalized autocorrelation function of the signals; calculate the value of τ corresponding to the maximal value of ρ(τ), and the reciprocal of the τ value is the pitch of the signals.
Step □ setting a low-pass or band-pass filter by using the pitch obtained by the pitch detection as cut-off frequency, and using the low-pass or band-pass filter to perform low-pass or band-pass filtering on corresponding high-pass filtered acceleration signals. After low-pass or band-pass filtering, relatively smooth signals can be obtained, to facilitate accurately calculating extreme value points of the acceleration signals. FIG. 6c is a signal graph, demonstrating low-pass filtered acceleration signals.

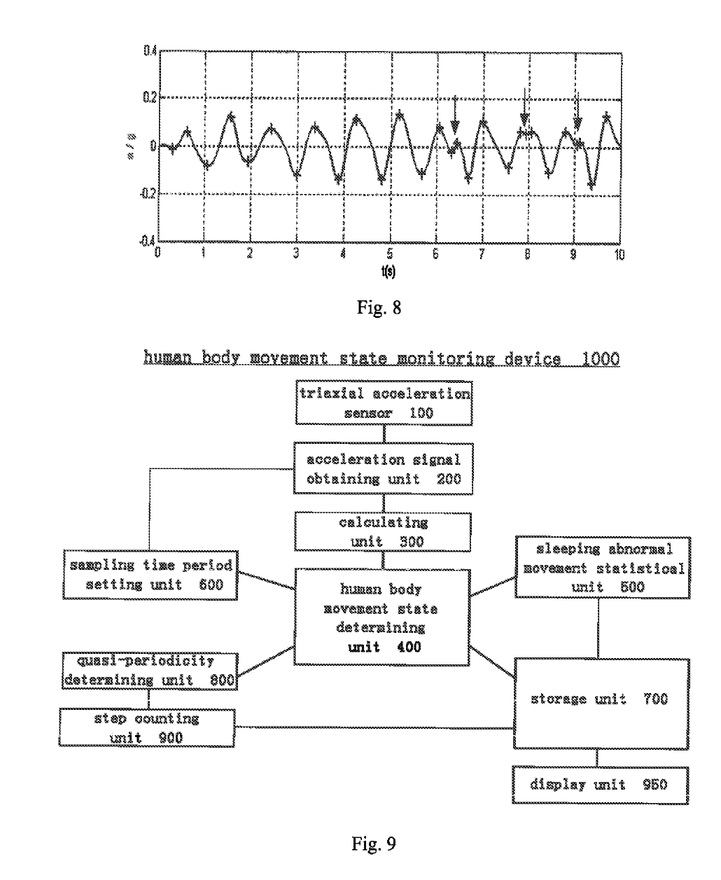

Step □ obtaining extreme value points of the acceleration signals in the low-pass or band-pass filtered acceleration signals and removing interfering extreme value points in the extreme value points of the acceleration signals, so as to obtain effective extreme value points. FIG. 6d is a signal graph, demonstrating an example of extreme value points of low-pass or band-pass filtered monoaxial acceleration signals, wherein, symbol + denotes extreme value point (comprising maximal and minimal value points). FIG. 6d demonstrates a rather specific example, wherein, noise interference is almost not existed in the low-pass or band-pass filtered acceleration signals. In a more general case, after low-pass or band-pass filtering, there still exists noise interference in the acceleration signals, which is represented by the existence of interfering extreme value points. FIG. 8 is a signal graph, demonstrating another example of extreme value points of low-pass or band-pass filtered acceleration signals. As shown in FIG. 8, interfering extreme value points (as indicated by arrow in FIG. 8) exists in the low-pass or band-pass filtered monoaxial acceleration signals, and these interfering extreme value points do not represent the extreme value points related to periodic movements, which only result in over-counting of step number, removing these interfering extreme value points will make the counted step number more accurate. Thus, it required to remove these interfering extreme value points, so as to obtain the extreme value point corresponding to walking and running step number accurately.

In one embodiment of the present invention, the interfering extreme value points may comprise such an extreme value point of the acceleration signals that the time gap between the extreme value point of the acceleration signals and the previous extreme value point of the acceleration signals is less than a predetermined threshold, wherein, the predetermined threshold is far less than the period of the pitch component of monoaxial acceleration signals. In the embodiment, in each group of extreme value points relatively close to each other, only one extreme value point on the leftmost is kept, and the other extreme value points are removed as interfering extreme value points. In this manner, the interfering extreme value points in the extreme value points of the acceleration signals are filtered out through the time gap between the extreme value points of the acceleration signals.

In another embodiment of the present invention, the interfering extreme value points may comprise such extreme value points of the acceleration signals, whose magnitude values are not maximal, among each group of extreme value points of the acceleration signals with time gaps continuously less than a predetermined threshold. In another words, in the embodiment, in each group of extreme value points relatively close to each other, only the extreme value point of the acceleration signals with maximal magnitude value is kept, and the other extreme value points are removed as interfering extreme value points. In this manner, the interfering extreme value points in the extreme value points of the acceleration signals are filtered out through the time gaps between the extreme value points of the acceleration signals and the magnitude values of extreme value points of the acceleration signals.

Step □ calculating time gaps between adjacent effective extreme value points, obtaining a time gap sequence, and calculating differences between adjacent time gaps in the time gap sequence, obtaining a time gap difference sequence, and if each of a continuous predetermined number of time gap differences in the time gap difference sequence is less than a predetermined period threshold, it is determined that the acceleration signals have quasi-periodicity, otherwise, it is determined that the acceleration signals do not have quasi-periodicity.

Returning back to FIG. 5, if the human body is in the irregular fierce movement state, then in step 85 accumulating the time periods of the acceleration signals into the total time period of the irregular fierce movement state, accumulating the energy of the acceleration signals into the total energy of the irregular fierce movement state, and setting the sampling time period of acceleration signals as the sampling time period of the fierce movement state, then returning to step S10. The sampling time period of fierce movement state may be 1˜3 seconds.

If the human body is in the regular fierce movement state, then in step S95 accumulating the time periods of the acceleration signals into the total time period of the regular fierce movement state, accumulating the energy of the acceleration signals into the total energy of the regular fierce movement state, calculating movement step number according to the acceleration signals, and accumulating the movement step number into the total movement step number, and setting the sampling time period of acceleration signals as the sampling time period of the fierce movement state, then returning to step S10.

The method of calculating movement step number according to the acceleration signals can comprise: count the effective extreme value points in the low-pass or band-pass filtered acceleration signals having quasi-periodicity, and the number of the effective extreme value points is the movement step number obtained in this step counting process. Furthermore, a displacement signal can be obtained by double integral of the acceleration signals on time, so as to provide reference for actual movement distance to a walker & runner. In addition, it can be distinguished whether it is in situ movements or actual walking & running according to the size of the displacement.

The human body movement state monitoring method of the present invention further comprises optionally displaying the total time period of the sleeping state, the total energy of the sleeping state, the total time period of the sleeping abnormal movements, the total time period of the light movement state, the total energy of the light movement state, the total time period of the irregular fierce movement state, the total energy of the irregular fierce movement state, the total time period of the regular fierce movement state, the total energy of the regular fierce movement state and the total step number of movement. Thus, according to these data, a person's sleeping quality, movement level and energy consumption condition can be known.

Hereinbefore the human body movement state monitoring method of the present invention is described by referring to FIGS. 1-8. The human body movement state monitoring method of the present invention can be achieved through software, and also can be achieved through hardware, or achieved in a combination way of software and hardware.

FIG. 9 is a block diagram, demonstrating a human body movement state monitoring device according to one embodiment. As shown in FIG. 9, a human body movement state monitoring device 1000 according to one embodiment of the present invention comprises: a triaxial acceleration sensor 100, an acceleration signal obtaining unit 200, a calculating unit 300, a human body movement state determining unit 400, a sleeping abnormal movement statistical unit 500, a sampling time period setting unit 600, a storage unit 700, a quasi-periodicity determining unit 800 and a step counting unit 900.

The acceleration signal obtaining unit 200 obtains acceleration signals having a set sampling time period from output of the triaxial acceleration sensor 100 worn on a human body, and the calculating unit 300 calculates the energy and average power of the acceleration signals.

The human body movement state determining unit 400 determines a human body movement state according to the average power of the acceleration signals, and if the average power of the acceleration signals is more than a predetermined fierce movement threshold, determines that the human body is in a fierce movement state; if the average power of the acceleration signals is less than a predetermined sleeping threshold, determines that the human body is in a sleeping state; if the average power of the acceleration signals is less than the fierce movement threshold and is more than the sleeping threshold, determines that the human body is in a light movement state;

If the human body movement state determining unit 400 determines that the human body is in the sleeping state, accumulates the time periods of the acceleration signals into the total time period of the sleeping state, accumulates the energy of the acceleration signals into the total energy of the sleeping state; the sleeping abnormal movement statistical unit 500 counts up the time periods of acceleration signals which have intensity more than a predetermined intensity threshold, and accumulates the counted time periods into the total time period of sleeping abnormal movements; the sampling time period setting unit 600 sets the sampling time period of acceleration signals as the sampling time period of the sleeping state; the storage unit 700 stores the total time period of the sleeping state, the total energy of the sleeping state and the total time period of the sleeping abnormal movements.

If the human body movement state determining unit 400 determines that the human body is in the light movement state, accumulates the time periods of the acceleration signals into the total time period of the light movement state, accumulates the energy of the acceleration signals into the total energy of the light movement state, and the sampling time period setting unit 600 sets the sampling time period of acceleration signals as the sampling time period of the light movement state; the storage unit 700 stores the total time period of the light movement state and the total energy of light movement state.

If the human body movement state determining unit 400 determines that the human body is in the fierce movement state, then the quasi-period determining unit 800 determines whether the acceleration signals have quasi-periodicity, and if determining that the acceleration signals do not have quasi-periodicity, then the human body movement state determining unit 400 determines that the human body is in an irregular fierce movement state, and if the quasi-period determining unit 800 determines that the acceleration signals have quasi-periodicity, then the human body movement state determining unit 400 determines that the human body is in a regular fierce movement state.

If the human body movement state determining unit 400 determines that the human body is in the irregular fierce movement state, then accumulates the time periods of the acceleration signals into the total time period of the irregular fierce movement state, accumulates the energy of the acceleration signals into the total energy of the irregular fierce movement state, and the sampling time period setting unit 600 sets the sampling time period of acceleration signals as the sampling time period of the fierce movement state; the storage unit 700 stores the total time period of the irregular fierce movement state and the total energy of the irregular fierce movement state.

If the human body movement state determining unit 400 determines that the human body is in the regular fierce movement state, then accumulates the time periods of the acceleration signals into the total time period of the regular fierce movement state, accumulates the energy of the acceleration signals into the total energy of the regular fierce movement state, and the step counting unit 900 calculates movement step number according to the acceleration signals, and accumulates the movement step number into the total movement step number; the sampling time period setting unit 600 sets the sampling time period of acceleration signals as the sampling time period of fierce movement state; the storage unit 700 stores the total time period of the regular fierce movement state, the total energy of the regular fierce movement state and the movement step number.

Preferably, the human body movement state monitoring device 1000 may further comprise a display unit 950 for optionally displaying the total time period of the sleeping state, the total energy of the sleeping state, the total time period of the sleeping abnormal movements, the total time period of the light movement state, the total energy of the light movement state, the total time period of the irregular fierce movement state, the total energy of the irregular fierce movement state, the total time period of the regular fierce movement state, the total energy of the regular fierce movement state and the total step number of movement.

Hereinbefore the human body movement state monitoring method and device according to the present invention are described by referring to attached drawings in an illustrative way. However, one skilled in the art should understand that, for the human body movement state monitoring method and device of the present invention, various modifications can be made without departing from the content of the present invention. Hence, the protection scope of the present invention should be determined by the content of appended claims.

It should be noted that:

The example of each component in the present invention can be achieved by hardware, or achieved by software module operating on one or more processors, or achieved in a combination thereof. Those skilled in the art should understand that, some or all functions of some or all components according to the example of the present invention can be achieved by using micro-processor or digital signal processor (DSP) in practice. The present invention can also be implemented as a program for carrying out part or all equipment or device in the described method herein (for example, computer program and computer program product). The program implementing the present invention as such can be stored on the computer readable medium, or may be having a form of one or more signals. Such a signal can be downloaded from internet website, or provided on a carrier signal, or provided in any other forms.

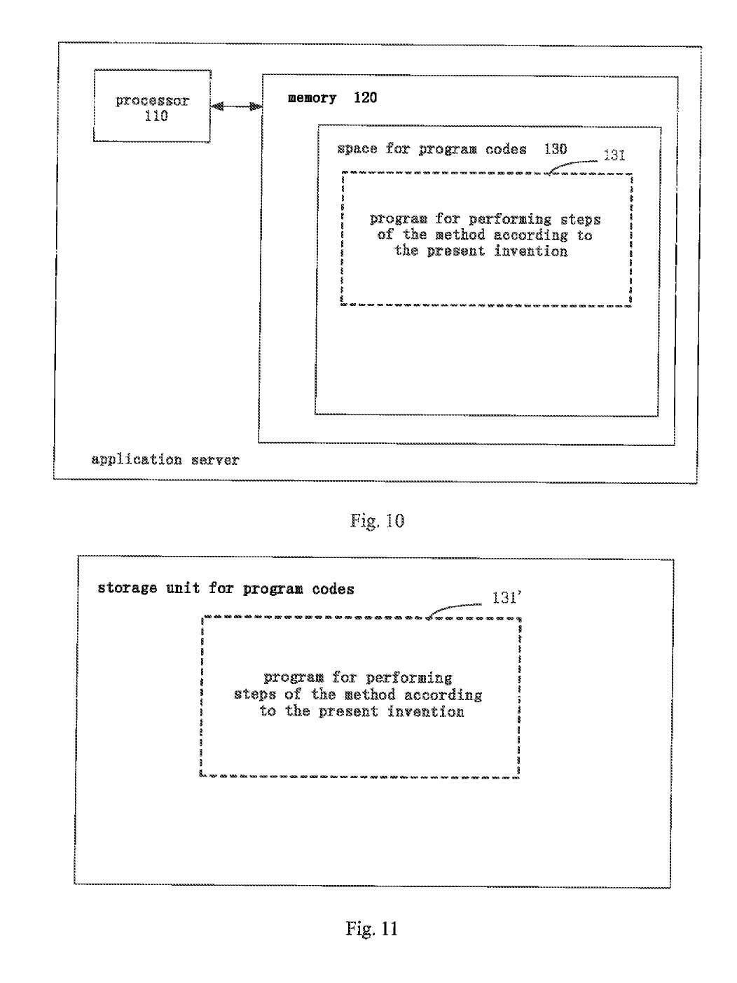

For example, FIG. 10 demonstrates a server capable of implementing the human body movement state monitoring method according to the present invention, e.g., an application server. The server conventionally comprises a processor 110 and a computer program product or computer readable medium in a form of memory 120. Memory 120 can be electronic memory, such as flash memory, EEPROM (electrically erasable programmable read only memory), EPROM, hard disk, ROM or the like. Memory 120 has a storage space 130 for the program codes 131 performing the steps of any method step in aforesaid methods. For example, the storage space 130 for program codes can comprise individual program codes 131 respectively for implementing each step in aforesaid method. These program codes can be read out from one or more computer program products or written into one or more computer program products. These computer program products comprise program code carriers such as hard disk, compact disk (CD), memory card or floppy disk and the like. Such computer program products generally are portable or fixed storage unit as shown in FIG. 11. The storage unit may have storage segment, storage space and the like set similar to that of the memory 120 in the server of FIG. 10. Program codes can be compressed in a suitable form. Generally, the storage unit comprises the computer readable codes 131′ for implementing the steps of the method according to the present invention, i.e., the codes can be read from processors such as 110 and the like, when these codes are operated by a server, the server is caused to carry out each step in aforesaid method.

It should be noted that the aforesaid examples are for explaining the present invention rather than limiting the present invention, and those skilled in the art can design alternative example without departing from the scope of appended claims. In the claims, any reference symbol located between parentheses should not be construed as limitation to claims. Word “comprise” does not exclude the existence of element or step not defined in the claims. The present invention can be implemented through hardware containing various different elements and through suitably programmed computer. In the device claim listing several devices, several of these devices can be specifically implemented by one same hardware item.

In the description provided herein, numerous specific details are described. However, it can be understood that, the examples of the present invention can be carried out without these specific details. In some examples, well-known methods, structures and techniques are not disclosed in details, so as not to blur the understanding of the present specification. The terms used in the present specification are mainly selected for the purpose of readability and instruction, rather than selected for explaining or limiting the subject matter of the present invention.

1. A human body movement state monitoring method, characterized in that, the method comprises the following steps performed repeatedly:
a) obtaining acceleration signals having a set sampling time period from output of a triaxial acceleration sensor worn on a human body, and calculating the energy and average power of the acceleration signals;
b) determining a human body movement state according to the average power of the acceleration signals, and if the average power of the acceleration signals is more than a predetermined fierce movement threshold, determining that the human body is in a fierce movement state, if the average power of the acceleration signals is less than a predetermined sleeping threshold, determining that the human body is in a sleeping state, if the average power of the acceleration signals is less than the fierce movement threshold and is more than the sleeping threshold, determining that the human body is in a light movement state;
c1) if the human body is in the sleeping state, accumulating time periods of the acceleration signals into a total time period of the sleeping state, accumulating the energy of the acceleration signals into a total energy of the sleeping state, counting up the time periods of acceleration signals which have intensity more than a predetermined intensity threshold, and accumulating the counted time periods into a total time period of sleeping abnormal movements, and setting a sampling time period of acceleration signals as a sampling time period of the sleeping state, then returning to step a);
c2) if the human body is in the light movement state, accumulating the time periods of the acceleration signals into a total time period of the light movement state, accumulating the energy of the acceleration signals into a total energy of the light movement state, and setting the sampling time period of acceleration signals as a sampling time period of the light movement state, then returning to step a);
c3) if the human body is in the fierce movement state, further determining whether the acceleration signals have quasi-periodicity, if the acceleration signals do not have quasi-periodicity, determining that the human body is in an irregular fierce movement state, if the acceleration signals have quasi-periodicity, determining that the human body is in a regular fierce movement state;
d1) if the human body is in the irregular fierce movement state, accumulating the time periods of the acceleration signals into a total time period of the irregular fierce movement state, accumulating the energy of the acceleration signals into a total energy of the irregular fierce movement state, and setting the sampling time period of acceleration signals as a sampling time period of the fierce movement state, then returning to step a);
d2) if the human body is in the regular fierce movement state, accumulating the time periods of the acceleration signals into a total time period of the regular fierce movement state, accumulating the energy of the acceleration signals into a total energy of the regular fierce movement state, calculating movement step number according to the acceleration signals, and accumulating the movement step number into a total movement step number, and setting the sampling time period of acceleration signals as the sampling time period of the fierce movement state, then returning to step a).

2. The human body movement state monitoring method according to claim 1, wherein the average power P of the acceleration signals is calculated from the following formula:
$$P = \frac{1}{N} {\sum }_{i = 1}^{N} {( {a}_{i} - {a}_{0} )}^{2}$$
wherein aᵢ is the No. i value of the acceleration signals, N is the length of the acceleration signals, and 1≦i≦N, a₀ is the average value of the acceleration signals,
$${a}_{0} = \frac{1}{N} {\sum }_{i = 1}^{N} {a}_{i} .$$

3. The human body movement state monitoring method according to claim 1, wherein the determining step of the quasi-periodicity of the acceleration signals comprises:
performing high-pass filtering on the acceleration signals;
performing pitch detection on the high-pass filtered acceleration signals;
setting a low-pass or band-pass filter by using the pitch obtained by the pitch detection as cut-off frequency, and using the low-pass or band-pass filter to perform low-pass or band-pass filtering on corresponding high-pass filtered acceleration signals;
obtaining extreme value points of the acceleration signals in the low-pass or band-pass filtered acceleration signals and removing interfering extreme value points in the extreme value points of the acceleration signals, so as to obtain effective extreme value points in the low-pass or band-pass filtered acceleration signals;
calculating time gaps between adjacent effective extreme value points, obtaining a time gap sequence, and calculating differences between adjacent time gaps in the time gap sequence, obtaining a time gap difference sequence, and if each of a continuous predetermined number of time gap differences in the time gap difference sequence is less than a predetermined period threshold, determining that the acceleration signals have quasi-periodicity, otherwise, determining that the acceleration signals do not have quasi-periodicity.

4. The human body movement state monitoring method according to claim 3, wherein the step of calculating movement step number according to the acceleration signals comprises:
counting the effective extreme value points in the low-pass or band-pass filtered acceleration signals having quasi-periodicity, the number of the effective extreme value points being movement step number.

5. The human body movement state monitoring method according to claim 4, further comprises obtaining a displacement signal by double integral of the acceleration signals on time.

6. The human body movement state monitoring method according to claim 3, wherein performing pitch detection on the high-pass filtered acceleration signals comprises:
attenuating the signals with a filter that attenuates signal energy with an incrementing degree from low frequency to high frequency;
obtaining the autocorrelation function ρ(τ) of the attenuated signals from the following formula:
$$ρ ( τ ) = \frac{{\sum }_{n = 1}^{N} a ( n ) a ( n - τ )}{\sqrt{{\sum }_{n = 1}^{N} {a}^{2} ( n ) {\sum }_{n = 0}^{N - 1} {a}^{2} ( n - τ )}}$$
wherein a(n) is the No. n value of the attenuated signals, N is the length of the signals, and 1≦n≦N, τ is a delay time, ρ(τ) is normalized autocorrelation function of the signals;
calculating the value of τ corresponding to the maximal value of ρ(τ), and the reciprocal of the τ value is the pitch of the signals.

7. The human body movement state monitoring method according to claim 3, wherein, removing the interfering extreme value points from the extreme value points of the acceleration signals comprises: filtering out the interfering extreme value points from the extreme value points of the acceleration signals through a time gap; alternatively, filtering out the interfering extreme value points from the extreme value points of the acceleration signals through a time gap and a magnitude value.

8. The human body movement state monitoring method according to claim 7, wherein the interfering extreme value points comprise such an extreme value point of the acceleration signals that the time gap between the extreme value point of the acceleration signals and the previous extreme value point of the acceleration signals is less than a predetermined threshold; or the interfering extreme value points comprise extreme value points of the acceleration signals, whose magnitude values are not maximal, among each group of extreme value points of the acceleration signals with time gaps continuously less than a predetermined threshold.

9. The human body movement state monitoring method according to claim 1, also comprises optionally displaying the total time period of the sleeping state, the total energy of the sleeping state, the total time period of the sleeping abnormal movements, the total time period of the light movement state, the total energy of the light movement state, the total time period of the irregular fierce movement state, the total energy of the irregular fierce movement state, the total time period of the regular fierce movement state, the total energy of the regular fierce movement state and the total step number of movement.

10. A human body movement state monitoring device, characterized in that, the device comprises: a triaxial acceleration sensor (100), an acceleration signal obtaining unit (200), a calculating unit (300), a human body movement state determining unit (400), a sleeping abnormal movement statistical unit (500), a sampling time period setting unit (600), a storage unit (700), a quasi-periodicity determining unit (800) and a step counting unit (900), wherein the acceleration signal obtaining unit (200) obtains acceleration signals having a set sampling time period from output of the triaxial acceleration sensor (100) worn on a human body, and the calculating unit (300) calculates the energy and average power of the acceleration signals;
the human body movement state determining unit (400) determines a human body movement state according to the average power of the acceleration signals, and if the average power of the acceleration signals is more than a predetermined fierce movement threshold, determines that the human body is in a fierce movement state, if the average power of the acceleration signals is less than a predetermined sleeping threshold, determines that the human body is in a sleeping state, if the average power of the acceleration signals is less than the fierce movement threshold and is more than the sleeping threshold, determines that the human body is in a light movement state;
if the human body movement state determining unit (400) determines that the human body is in the sleeping state, accumulates the time periods of the acceleration signals into a total time period of the sleeping state, accumulates the energy of the acceleration signals into a total energy of the sleeping state; the sleeping abnormal movement statistical unit (500) counts up the time periods of acceleration signals which have intensity more than a predetermined intensity threshold, and accumulates the counted time periods into a total time period of sleeping abnormal movements; the sampling time period setting unit (600) sets the sampling time period of acceleration signal as the sampling time period of the sleeping state; the storage unit (700) stores the total time period of the sleeping state, the total energy of the sleeping state and the total time period of the sleeping abnormal movements;
if the human body movement state determining unit (400) determines that the human body is in the light movement state, accumulates the time periods of the acceleration signals into a total time period of the light movement state, accumulates the energy of the acceleration signals into a total energy of the light movement state, and the sampling time period setting unit (600) sets the sampling time period of acceleration signals as the sampling time period of the light movement state; the storage unit (700) stores the total time period of the light movement state and the total energy of light movement state;
if the human body movement state determining unit (400) determines that the human body is in the fierce movement state, then the quasi-period determining unit (800) determines whether the acceleration signals have quasi-periodicity, and if determining that the acceleration signals do not have quasi-periodicity, then the human body movement state determining unit (400) determines that the human body is in an irregular fierce movement state, and if the quasi-period determining unit (800) determines that the acceleration signals have quasi-periodicity, then the human body movement state determining unit (400) determines that the human body is in a regular fierce movement state;
if the human body movement state determining unit (400) determines that the human body is in the irregular fierce movement state, then accumulates the time periods of the acceleration signals into a total time period of the irregular fierce movement state, accumulates the energy of the acceleration signals into a total energy of the irregular fierce movement state, and the sampling time period setting unit (600) sets the sampling time period of acceleration signals as the sampling time period of the fierce movement state; the storage unit (700) stores the total time period of the irregular fierce movement state and the total energy of the irregular fierce movement state;
if the human body movement state determining unit (400) determines that the human body is in the regular fierce movement state, then accumulates the time periods of the acceleration signals into a total time period of the regular fierce movement state, accumulates the energy of the acceleration signals into a total energy of the regular fierce movement state, and the step counting unit (900) calculates movement step number according to the acceleration signals, and accumulates the movement step number into a total movement step number; the sampling time period setting unit (600) sets the sampling time period of acceleration signals as the sampling time period of fierce movement state; the storage unit (700) stores the total time period of the regular fierce movement state, the total energy of the regular fierce movement state and the movement step number.

11. The human body movement state monitoring device according to claim 10, further comprising a display unit for optionally displaying the total time period of the sleeping state, the total energy of the sleeping state, the total time period of the sleeping abnormal movements, the total time period of the light movement state, the total energy of the light movement state, the total time period of the irregular fierce movement state, the total energy of the irregular fierce movement state, the total time period of the regular fierce movement state, the total energy of the regular fierce movement state and the total step number of movement.

12. (canceled)

13. (canceled)
